(19) United States
(12) Patent Application Publication
(10) Pub. No.: US 2002/0082466 A1
(43) Pub. Date: Jun. 27, 2002

(54) Laser surgical system with light source and video scope

(76) Inventor: Jeongho Han, Seoul (KR)

Correspondence Address: Maine & Asmus, P.O. Box 3445, Nashua, NH, 03061-3445, US

(21) Appl. No.: 09/747,732

(22) Filed: Dec. 22, 2000

(51) Int. Cl. A61B 18/20

(52) U.S. Cl. 600/13; 606/10; 600/19

(57) ABSTRACT

An apparatus for delivering a laser beam to a surgery situs while providing a live video presentation of the surgery situs. The present invention comprises a laser source, preferably CO2, which is attached to an articulated arm having a zoom assembly. A laser head assembly has an adjustable reflection mirror for directing the laser beam to the surgery situs, and a camera with a plurality of lenses to capture the image of the surgery situs. The reflection mirror lies within in the path of the camera lens, but by adjusting the focal point lenses, the image is captured and displayed on a monitor. A light source illuminates the surgery site to provide sufficient lighting for the image capture.

### BACKGROUND OF THE INVENTION

1. Technical Field of the Invention

This invention most generally relates to a laser surgery device, and more particularly for a laser surgery apparatus operating simultaneously with a video system.

2. Background of the Invention

Lasers are successfully employed in many industries, and have been proven to be reliable within the medical community as well. Laser surgery is widely accepted and practiced and lasers are used in almost every medical field for performing a variety of surgical and non-surgical procedures.

A laser provides a source of light of one pure color or wavelength, which can be focussed to a very small spot. It can also shine for long distances without spreading out or diverging. With respect to laser surgery the laser light is invisible, requiring a very clear image of the surgery area to carefully monitor the surgery. The precise beam of a laser supplies sufficient energy to perform controlled surgical procedures that far exceed the capabilities of scalpel and other manual surgical procedures.

The energy of a laser comprises light photons that are absorbed by tissue. In laser surgery, the wavelength of the laser emission is chosen to correspond to the preferential absorption band of an imbedded tissue. The amount of energy absorbed by the tissue depends on the color or wavelength of the laser light and the color and composition of the tissue. If the color absorbed by the tissue (all colors except the color of the tissue that is reflected) matches the laser color then a lot of energy will be absorbed and vice versa. By selecting the proper wavelength to effect the underlying tissue, a laser can perform non-invasive surgery without perforating the overlying tissue. The energy supplied by the laser is also used to cut and coagulate or to destroy tissue.

Although non-invasive surgery is possible, it is often very difficult, as tissue is generally not uniformly distributed. There is however general agreement in the medical profession that efforts should be taken to minimize the laser energy deposition on living tissues in front and behind the targeted tissue. This requires a clear and undisturbed view of the surgery situs during the entire surgical procedure.

Lasers deliver energy to the surgical target area at a high intensity and at a very precise location. In certain surgical procedures laser light is transmitted to the surgery area by using an endoscope, where light was delivered through optical fibers to illuminate the area. An endoscope is basically a small tube that enters through any opening such as the penis for a prostate operation or through a small incision in the abdomen for a gall stone operation. Endoscopes probe into areas such as the stomach, pancreas, colon, ear, nose and sinuses. Typically fiber optics is used to illuminate the area, as the light passes down the very thin glass optical fiber that resides within the endoscope.

Endoscopes are very flexible and can be up to about two meters in length. Some endoscopes have separate channels for obtaining an image of the surgery area, such as an image guide that contains thousands of optic fibers, wherein the optic fibers were used to provide an image of the surgery area. The resolution of the image was dependent upon several factors including having a large number of fibers within the endoscope.

Other prior art systems use articulated arms with extensive waveguides and reflective mirrors. However it is very difficult to use CO2 laser with an articulated arm, because it is difficult to view the exact area to be treated in the small canal such as the ear, nose, and throat. It is also difficult because the CO2 laser beam in the articulated arm delivery has to be changed at certain angles with reflection mirrors in order to deliver the laser beam to the treated area.

In the case of myringotomy ear surgery for example, a laser is used for fenestration of the tympanic membrane. A precision laser cut is made in the eardrum using an otoscope, or hand held laser delivery system. Using the prior art systems, the laser beam is not accurately delivered to the tympanic membrane without damaging other tissue in the ear and requiring longer healing periods. A CO2 laser is preferred for myringotomy surgery along with a video display system to view the surgery area while performing the surgery. This requires a quality video camera and sufficient lighting of the target area.

Current endoscopes use a charged coupled device (CCD) camera that resides on the end of the endoscope. The technology related to the CCD camera has vastly improved over the last few yeas producing very small devices that provide clear images. The image data passes from the CCD to a video processing system that displays the image with great resolution.

One of the key elements of laser surgery is to have an exceptional view of the surgery situs while using a high precision laser to perform the surgical procedure. Therefore an optical imaging system is needed concurrently with a laser delivery system. The coordination and implementation of the imaging system and the laser system is complex, expensive, and difficult to maintain in a calibrated state.

In general, prior art systems tried two different methods for capturing an image simultaneously with the surgery. The first method used different channels for the laser and the imaging system, where the imaging system has an image capture and light source. These systems generally use flexible guided lasers such as ND-Yag lasers because the cap at the end of the laser delivery system must be small while providing dual channels at the target area. The second method uses a dichroic beam combiner to pass the imaging light and the laser beam at the same time.

To alleviate some of the difficulties, improved video scope systems are needed to see inside of the ear, nose, and throat during surgical procedures. However, the prior art systems use other special instrument while monitoring these areas with increased cost and complexity. It is well documented that using CO2 laser to treat the target area results in more accurate surgey, less damage to surrounding tissue, and faster healing time.

Many attempts have been made to address the aforementioned problems. In the prior art generally, there were various laser systems with mirrors and some have articulated arms. There are also video scope systems that may have a mirror. Some video scopes are also used in conjunction with lasers. Units that combine laser surgery with video scope capability have tried to use separate channels to separate the laser beam path from the video scope paths. Other systems use a series of mirrors and dichroic beam combiners to pass the laser and light as the same time. Such prior art systems are rather complex and have higher costs because of the additional manufacturing and component costs. Additionally, the present laser devices are generally expensive instruments with complicated elements and procedures for operation. The complex operating steps add time to the surgery that is reflected in the cost of the operating surgery.

In U.S. Pat. No. 4,917,083, a laser system employing an articulated arm with a flexible waveguide section to direct the laser beam from the stationary CO2 laser source and through the articulated arm to the area of interest. This invention describes the basic concept of laser delivery systems, although no imaging system is incorporated.

A laser surgery invention is illustrated in U.S. Pat. No. 5,709,677 and related PCT patent application WO 99/55218, wherein an articulated arm extends to the operating region with a housing having a double wall with separated channels. An imaging system utilizes the separate channel, and sends images to a viewing device. The laser is illuminated through the housing structure via mirror(s), while the housing is temporarily affixed to the patient's head. The laser device described in U.S. Pat. No. 5,893,828 also discloses a delivery device that uses multiple channels for separating the laser system from the imaging system.

U.S. Pat. No. 5,198,926 describes an optical system that uses a first wavelength to focus the precise location as a visible marker and a second wavelength of laser light for surgical purposes. The system employs multiple lenses to function and uses a micromanipulator to extend the laser to the situs. The primary emphasis of this patent is beam alignment between the guide beam and the CO2 laser beam. A related patent is U.S. Pat. No. 6,091,074, wherein a camera is used in conjunction with a laser for precise location alignment. This invention is a beam alignment method using a target detection camera, and uses the camera to locate the target area and locating the laser beams active area—and adjusting the laser beam using the video images as a guide.

In U.S. Pat. No. 5,364,390 ('390) a complex laser surgical system is disclosed that uses an articulated arm to extend the laser hand piece to the surgery situs. The hand piece uses prisms to deliver the laser beams and a control system for signal processing. This patent is specifically detailed for eye and dental surgery using a Neodymium (ND) Yag laser. The '390 invention uses a diachronic reflector to reflect the laser beam from one direction and to capture an image from the other direction. This invention uses the ND-Yag laser because a CO2 laser requires metallic coated reflection mirrors such as zinc or gold, and these coatings do not allow the capture of the image from the other direction. In order to get the focused output beam, the '390 employs negative and positive lenses that are moveable at the end of the hand piece.

A video scope system is illustrated in U.S. Pat. No. 5,159,920 ('920) that demonstrates the usage of a video camera and fiber optics to generate a screen display of the internal situs. This invention shows one embodiment of providing illuminating light for the camera. The '920 patent is tailored to urology and for a flexible waveguide such as the ND-Yag laser or diode laser. There are two channels, one for the instruments and the flexible laser, and one for deploying the image lens and fiber optics.

European patent application EP 512,292A1 demonstrates a laser video scope system that uses an endoscope for both illuminated viewing and laser surgery. The viewing images are supplied by the optical fibers located in close proximity to the laser fibers.

Ideally, what is needed is a hand held laser surgical device that allows simultaneous video display and precision laser delivery. Such a system should be attached to an articulated arm to maintain the laser in a stable position. This device should not rely upon complex arrays of prisms and mirrors to provide a light source and imaging, nor should it incorporate multiple channels for separated imaging and laser delivery. Such a device should be cost effective to manufacture and simple to operate.

### SUMMARY OF THE INVENTION

The present invention is an apparatus for the delivery of precision laser beams to a target site with a simultaneous video display of the target site. More particularly, a CO2 laser beam and sufficient lighting are delivered to a specific area and monitored through currently available video scopes, such as a standard charge coupled device (CCD) camera. The laser beam and the light source/reflections occupy the same channel, but the present system allows viewing of the laser surgery site while the laser is in operation. This is accomplished by having an adjustable mirror assembly for reflecting the laser beam, a light source, and using a camera and lens that has an adjustable focal point and captures the surgery site while avoiding interference from the laser reflecting mirror.

The present invention is a surgical laser system having an articulated arm extending to a laser head. The laser head has a disposable cap and employs a video scope with a separate light source for illumination. There is a laser mirror with adjustment means, wherein the laser beam reflects off the mirror and the user adjusts the mirror to focus the laser onto the surgery area. The laser mirror is suspended within the laser head and supported by wires. The light source is used to illuminate the area of interest for the video scope system. A disposable cap is used wherein the shape of the disposable cap is dependent upon the desired target area such as ear, nose and throat.

One of the features of the present invention is the ability to capture live images of the surgery situs without separate imaging channels. The present invention uses lens theory to retrieve the image data even though the reflection mirror is dangling from the hanging wire in front of the camera lens.

It is an object of the present invention to provide a simplistic and cost effective delivery devices with demanding tolerances while providing high-precision laser surgery with live images in real time.

The present invention does not employ separate channels of dichroic beam combiners to function, thereby lowering cost and complexity. The present invention is particularly suited to treat ear, nose, and throat problems.

An object of the invention is a laser beam delivery apparatus with a video display system, comprising a housing for the laser beam delivery apparatus, wherein the housing has a front section, a center section, and a rear section. The laser source is used for generating a laser beam, wherein the laser source interconnects to the center section. There is a means of directing the laser beam to the target area and there is a light source for illuminating the target area. The invention has a means for capturing an image, wherein the means for capturing is located in the rear section and has an adjustable focal point within the center section. The captured is presented on a display monitor providing the video display of the target area.

A further object is a laser beam delivery, further comprising a zoom assembly for controlling the laser beam. Furthermore, a preferred laser source is a CO2 laser.

Another object is a laser beam delivery apparatus, further comprising a disposable cap on the forward section, wherein the cap has a narrow profile.

And another object is a laser beam delivery apparatus, wherein the means for capturing is a charge coupled device. In addition, the present invention further comprises an articulated arm connecting the laser source to the center section.

An object includes a laser beam delivery apparatus, wherein the means of directing the laser beam is a reflection mirror. And, wherein the reflection mirror is adjustable.

An object is a laser beam delivery apparatus, wherein the means for capturing comprises a plurality of lenses and a camera.

Also an object is a laser beam delivery apparatus, wherein the light source is a bulb, or a plurality of optical fibers.

An object of the invention is a laser beam delivery apparatus with a video display system, comprising a housing containing a laser reflection section, an image capture section, and an illumination means, wherein the illumination means provides illumination to a target area. There is a laser source for generating a laser beam, wherein the laser source interconnects to the laser reflection section and the laser beam is directed to the target area. In addition, there is a camera and one or more lenses within the image capturing section with an adjustable focal point to produce an output product of the target area while operating the laser.

A further object includes a laser beam delivery apparatus, wherein the adjustable focal point is adjusted by moving the camera. Furthermore, wherein the output product is a video display.

Yet another object is a laser beam delivery apparatus, further comprising a reflection mirror for directing the laser beam, wherein the reflection mirror is suspended by wires, and wherein one of the wires is adjustably connected to a manual control.

An object of the invention is a method of performing a laser surgery procedure with simultaneous video display comprising the steps of turning on power to a light source and a video monitor, and also powering a laser source to a low-power level. The invention includes extending an articulated arm to move a laser head in close proximity to a surgery target area, adjusting a focal point of the target area, capturing the video display of the target area with a camera, rotating an adjustment knob of a reflection mirror to direct a laser beam to the target area, focusing the laser beam and adjusting power of the laser power source for the surgery procedure.

And a final object is a method of adjusting the focal point by moving the camera, and wherein the focal point is adjusted forward of the reflection mirror.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

Still other objects and advantages of the present invention will become readily apparent to those skilled in this art from the detailed description, wherein we have shown and described only a preferred embodiment of the invention, simply by way of illustration of the best mode contemplated by us on carrying out our invention. As will be realized, the invention is capable of other and different embodiments, and its several details are capable of modifications in various obvious respects, all without departing from the invention.

### BRIEF DESCRIPTION OF THE DRAWINGS

FIG. 1 side view representation showing the laser source, articulated arm, laser head, and connections to the light source and video monitor

FIG. 2 laser beam delivery apparatus using any type of video camera

FIG. 3A disposable caps for ear canal

FIG. 3B disposable caps for ear canal

FIG. 3C disposable caps for nose

FIG. 3D disposable caps for throat

FIG. 4A sectional view of zoom assembly for adjusting laser beam size coming from laser source and through the articulated arm and reflection mirror housing with elements to change the direction of laser beam using adjusting knob

FIG. 4B exploded view of adjustment means for reflection mirror assembly

FIG. 5A sectional view of lenses, camera and light source using illuminating fibers

FIG. 5B sectional view showing camera adjustment

FIG. 6A side view of illumination connector

FIG. 6B front view of illumination connector

FIG. 7 illumination by means of optical fibers located near lenses

FIG. 8 illumination by means of one or more bulbs located near lenses

FIG. 9 illumination by means of optical fibers in front section

FIG. 10 illumination by means of one or more bulbs in front section

FIG. 11 illustration of image capture with reflection mirror located rearward of focal point

FIG. 12 illustration of image capture with reflection mirror located at focal point

FIG. 13 illustration of image capture with reflection mirror located forward of focal point

### DESCRIPTION OF THE PREFERRED EMBODIMENT

To those skilled in the art, the invention admits of many variations. The following is a description of a preferred embodiment, offered as illustrative of the invention but not restrictive of the scope of the invention. This invention makes it possible to deliver a CO2 laser beam into a small area while closely monitoring the area on a display. The present invention permits the user to treat the exact area using the CO2 laser head and utilizing any current video scope system. These novel aspects of the present invention will be discussed in terms of several scenarios that demonstrate various embodiments of the invention.

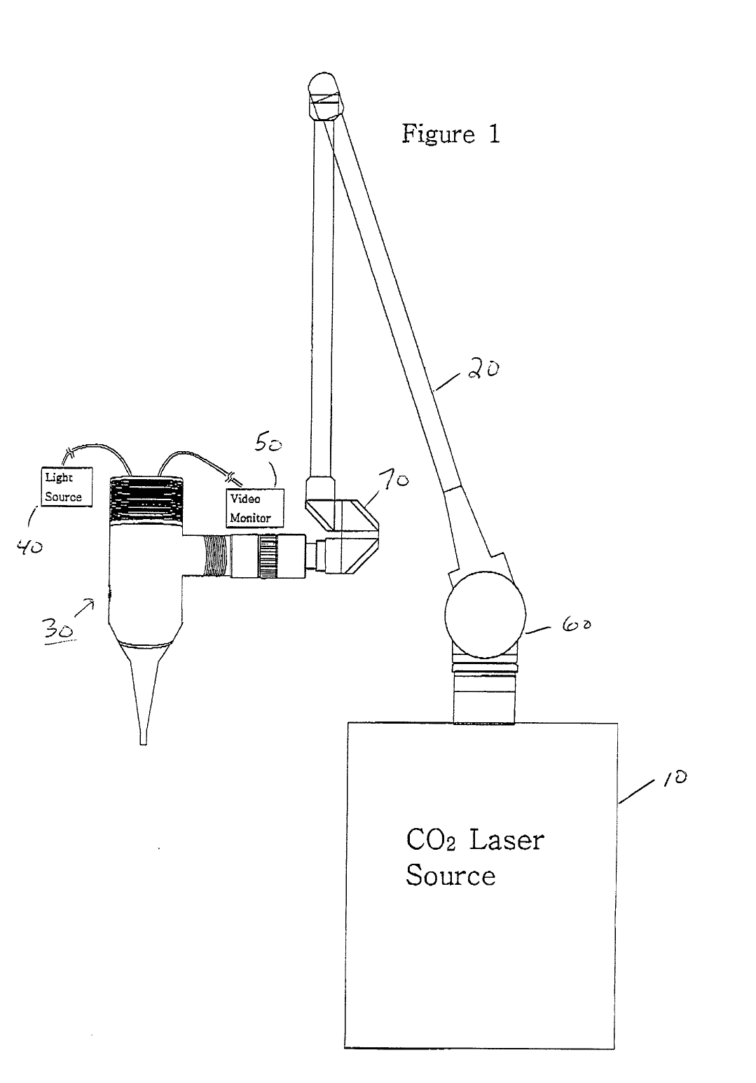

As shown in FIG. 1, a CO2 laser source 10 has an articulated arm 20 connecting therefrom, permitting the articulated arm 20 to extend in many different directions and at required distances for performing surgical procedures. The articulated arm 20 connects to the laser source 10 at a swivel ball base 60 that allow the arm 20 to rotate freely about the base 60. At the opposing end of the articulated arm 20 is a further swivel connection 70 that permit the attached laser head 30 to accommodate various positions. Such articulated arms are common in the industry. Connected or otherwise attached to the laser head 30 is a light source 40 and a video monitor 50.

The light source 40 is used to illuminate a target in the front of the laser head 30. In FIG. 1 the light source 40 is shown in the rear of the head 30, but can be introduced at any point within the head 30 and by several different manners as explained herein.

Although the video or display monitor 50 is shown connected to the output of the unit, the information from the laser head 30 is standard output data that is easily converted into other forms and can go other devices such as printers, plotters, or otherwise transmitted or stored as electronic data.

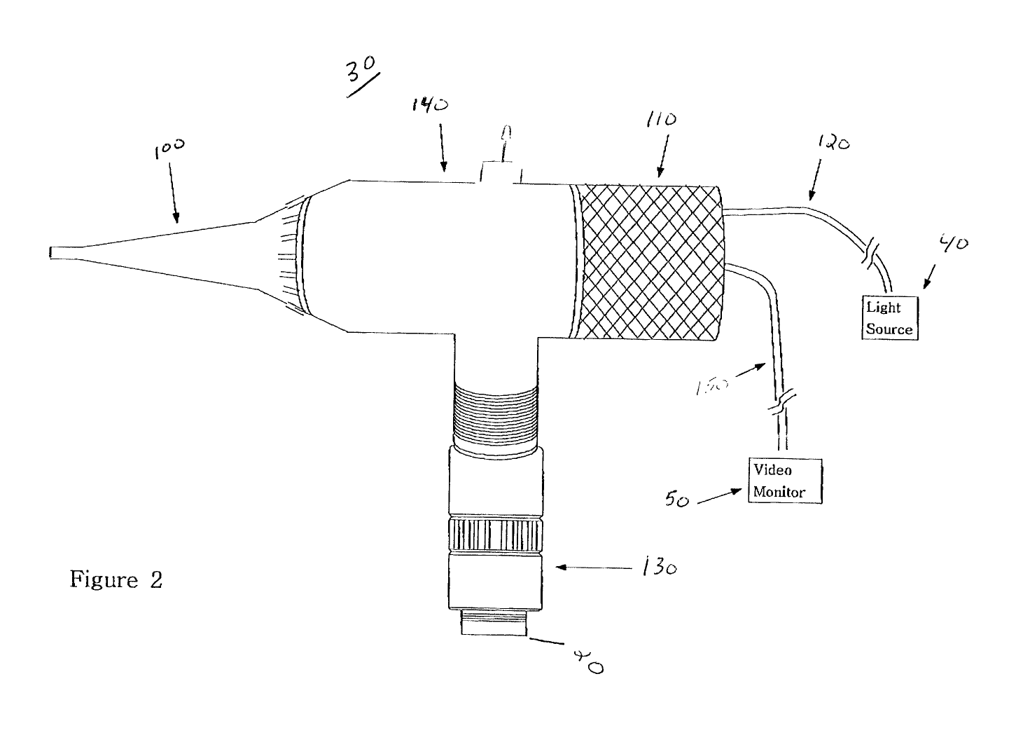

A more detailed side view of the apparatus for delivering the laser beam is shown in more detail in FIG. 2. The laser head 30 is a gun-shaped unit with the zoom assembly 130 forming the handle. The zoom assembly 130 for the CO2 laser beam is connected between the articulated arm 20 and the laser head 30, and controls the laser beam spot size. The zoom assembly 130 has a rotatable collar that adjusts the beam diameter of the laser and focuses the laser beam.

The middle section 140 of the laser head 30, also called the reflection mirror housing, contains the elements used to direct the laser beam to the disposable cap 100 and to the target area. The rear end 110 of the laser head 30 is referred to as the imaging section, and contains the camera and lens unit. Also illustrated are a light source connection 120 to a light source 40 and a video connection 150 to a video monitor 50.

A funnel-shaped disposable cap 100 extends from a front end of the laser head 30 and is used to place the unit in close proximity to the surgery situs. The shape of the disposable cap100 is intended for the type of surgery required. These particular caps are intended for the ear, nose, and throat, and are disposed of when finished.

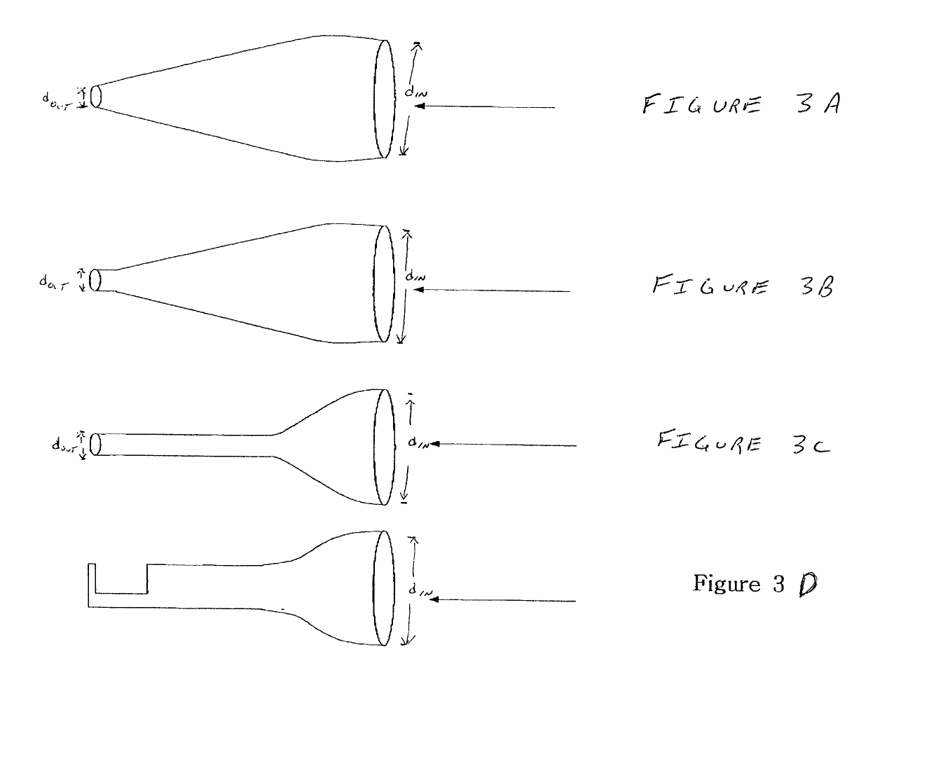

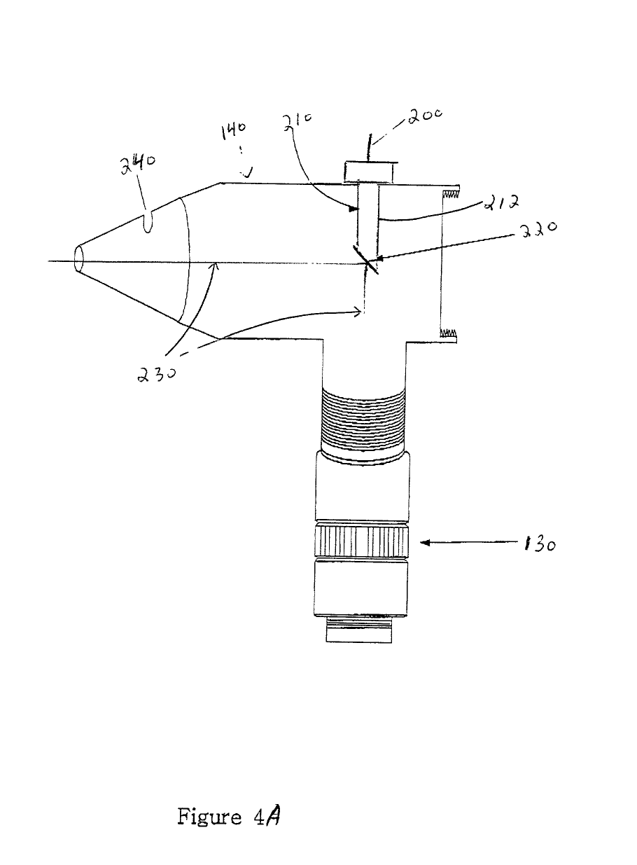

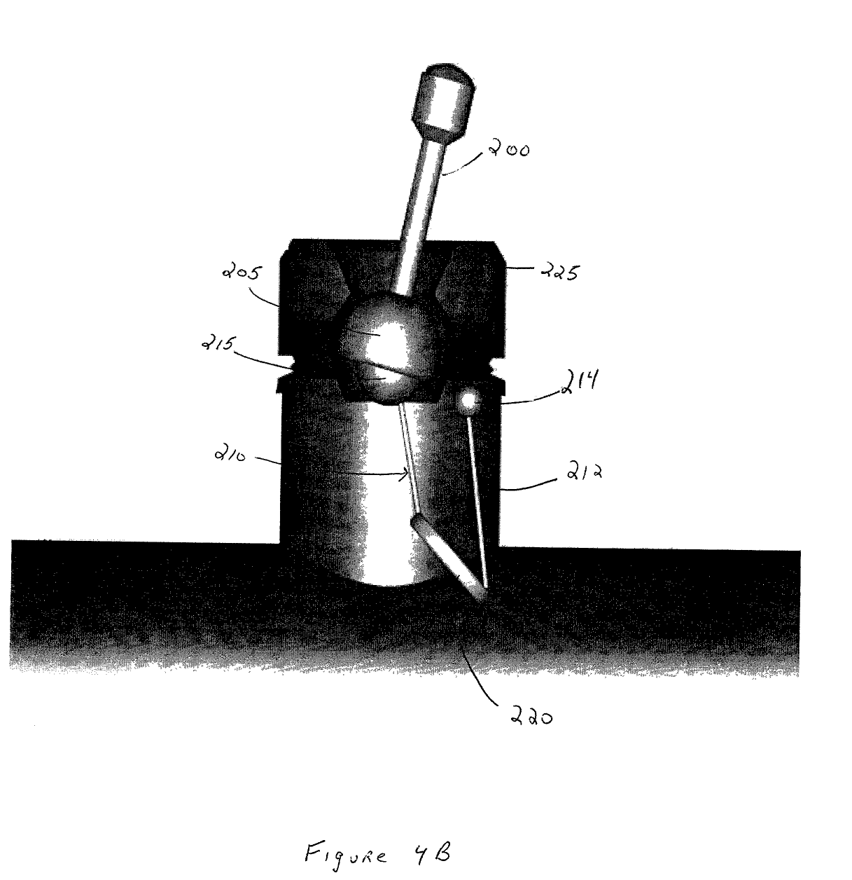

FIGS. 3A, 3B, 3C and 3D illustrate some of the various sizes and shapes of the disposable caps 100. The design of the cap aids in projecting the laser beam to the precise location of the laser surgery. FIGS. 3A and 3B shows disposable caps used for the ear canal in order to remove polyps in the ear or to fenestrate the tympanic membrane. FIG. 3C illustrates a disposable cap used for the nose, and FIG. 3D displays a disposable cap used for the throat. By designing the system without separate channels for the laser delivery and imaging functions, the caps have a more narrow profile that are possible with the systems having both a laser channel and an illumination channel. The smaller profile of the caps are easier to work with and provide greater visibility around the cap to observe initial positioning and penetration. It is also useful to be positioned in places not accessible by larger caps. In particular, the input diameter dᵢₙ mates with the laser head 30, and is smaller than conventional units because there is a single channel.

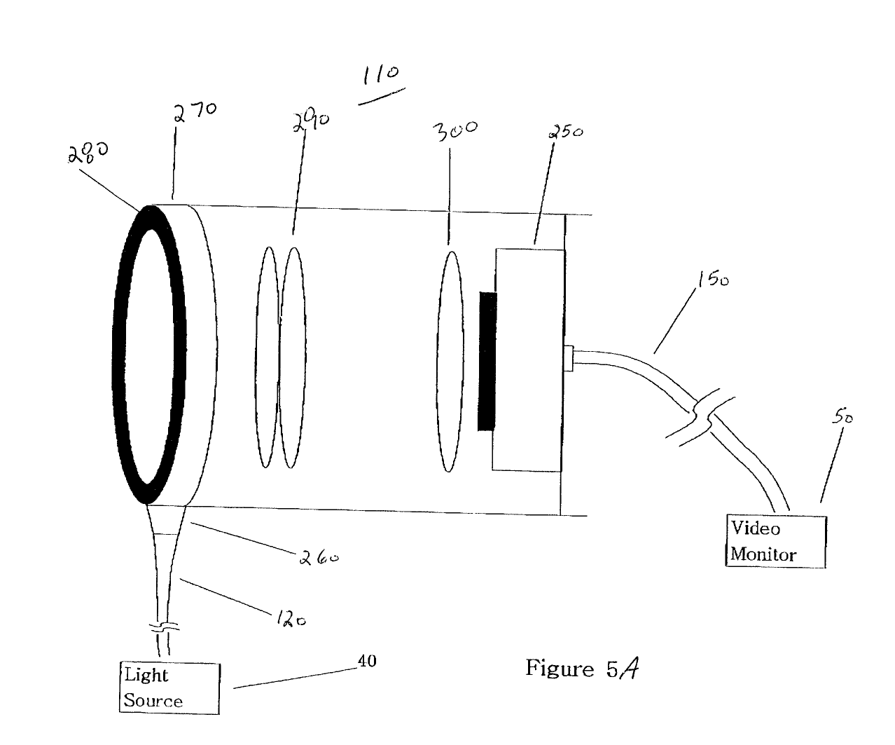

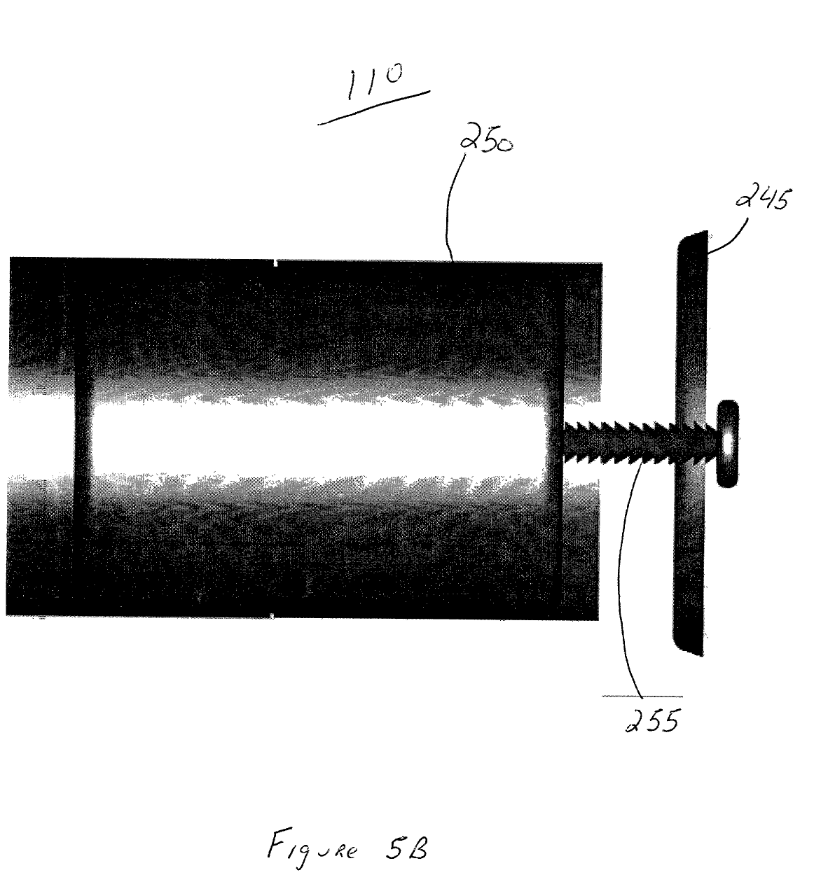

FIG. 4A shows the elements contained within the reflection mirror housing 140. A reflection mirror 220 is connected to an adjustable hanging wire 210, and a fixed wire 212, with an adjustment knob 200 that permits manual adjustment of the mirror 220. The laser beam 230 comes from the laser source (not shown) and is adjusted by the zoom assembly 130 to focus the beam 230. The adjusted beam 230 goes to reflection mirror 220, and the mirror 220 changes the beam direction approximately 90 degree from the original direction. The direction of the laser beam 230 is adjustable using the adjusting direction knob 200. The retaining slot 240 mates with the disposable cap 100 (not shown) to securely retain the disposable cap 100.

There are several methods for adjusting the mirror that are within the scope of the invention. The goal of the adjustment means is to ensure the laser beam 230 is reflected through the opening in the laser head 30. An exploded view of one embodiment of an adjustable reflection mirror assembly is shown in FIG. 4B. The adjustment stick 200 protrudes from an adjustment section housing 225 and is easily grasped by a user. The adjustment stick 200 rests atop a collar 205, wherein the stick 200 is a threaded member and screws into the collar 205. The collar 205 is retained within the adjustment housing 225 and adjustably connects to an adjustment ball 215. In direct contact with the adjustment ball 215 is a moveable wire 210, providing an adjustment mechanism using the adjustment knob 200 to influence the reflection mirror 220.

By moving the adjustment knob 200, the user controls the angle of the reflection mirror 220, so the user can adjust and direct the laser beam 230 through the disposable cap 100 to the target area. In a preferred embodiment, the reflection mirror 220 has a fixed wire 212 connected to the rearward portion of the reflection mirror 220. This fixed connection provides a pivot point for the adjustment and maintains the reflection angle from being manipulated to an adjustment outside possible bounds. The fixed wire 212 is connected to a captivated ball 214 that is retained within the housing 225. The adjustment is relatively small, so the adjustment mechanism is a fine tune adjustment. Once the proper angle of the reflection mirror is achieved, the stick 300 is screwed down by the user to retain the ball 215 captive within the collar 205 and lock the reflection angle.

In the illustrated embodiment, the adjusting wire 210 and the fixed wire 212 are steel wires of about 0.01 mm, however other guages of wire and different materials for the wire are contemplated by the inventors and within the scope of the invention. Transparent or semi-transparent materials are considered because they further decrease any interfere with the video display.

One embodiment showing the components making up the imaging section 110 of the laser head 30 is detailed in FIG. 5A. The light source 40 provides the lighting needed by the camera 250. The light cable 120 delivers the light to a light connector 260 from the light source 40. The light connector 260 is inter-connected to the illumination connector 270 that uses fiber optics 280 to provide the lighting within the assembly. The illumination connector 270 provides lighting for the target area and is reflected from the target area. The reflected light from the target area is collected by the camera lens(es) 290, 300 and the image is captured by the camera 250. In a preferred embodiment the reflected light travels through a pair of camera lenses 290 and also through an additional lens assembly 300 before being captured by the camera 250. The captured image is transmitted via the image cable 150 to the video monitor 50 or to other display, print, or storage mediums. Lens theory related to photography and cameras is well-known in the art and the number, thickness, and type of lenses 290, 300 are those understood by those skilled in the art.

The means of adjusting the focal point for viewing the object is to either move the camera or move the lenses. In a preferred embodiment the camera 250 is adjustable by turning a threaded member 255 that is interacts with a threaded plate 245 of the housing 110. The threaded member pushes or pulls the camera to the optimal focal point. It would be obvious to those skilled in the art that other forms of adjusting the camera or lens have been contemplated as means of adjusting the focal point. For example, the lenses could be slidably adjusted within the housing 110 along a track or slide.

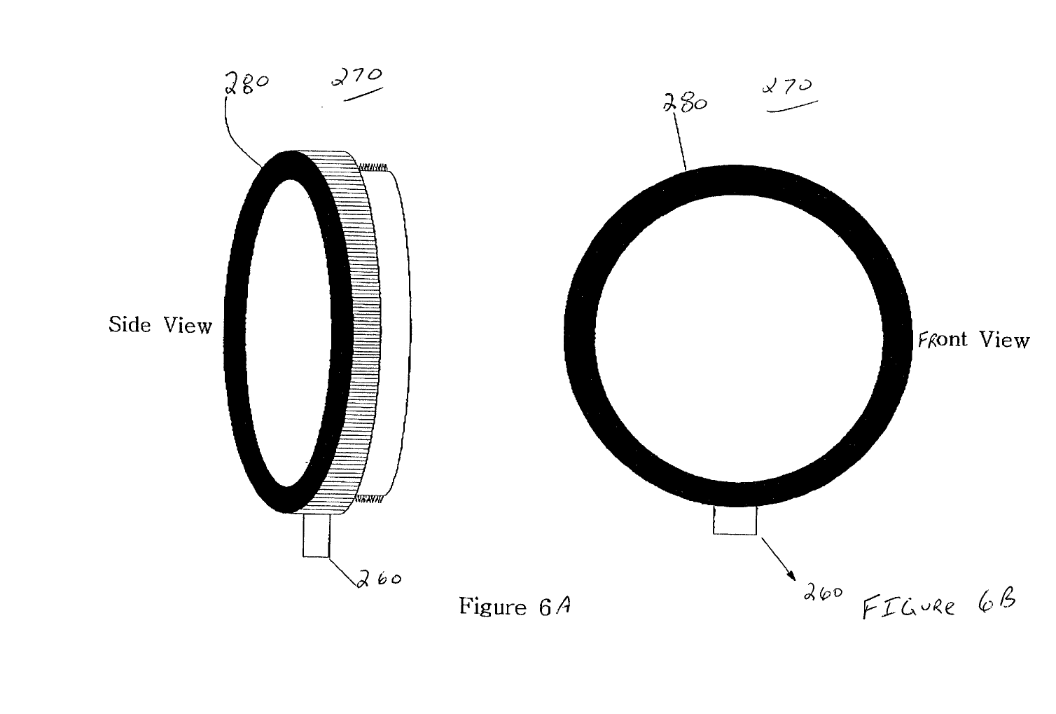

The illumination connector 270 shown in FIG. 5A is further illustrated in FIGS. 6A and 6B, which shows the front view and side view respectively. The illumination connector 270 has illuminated optical fibers 280 that receive light source via the light connector 260 that connect to a light source 40 (not shown). The optical fibers 280 provide the illumination directed towards the target area.

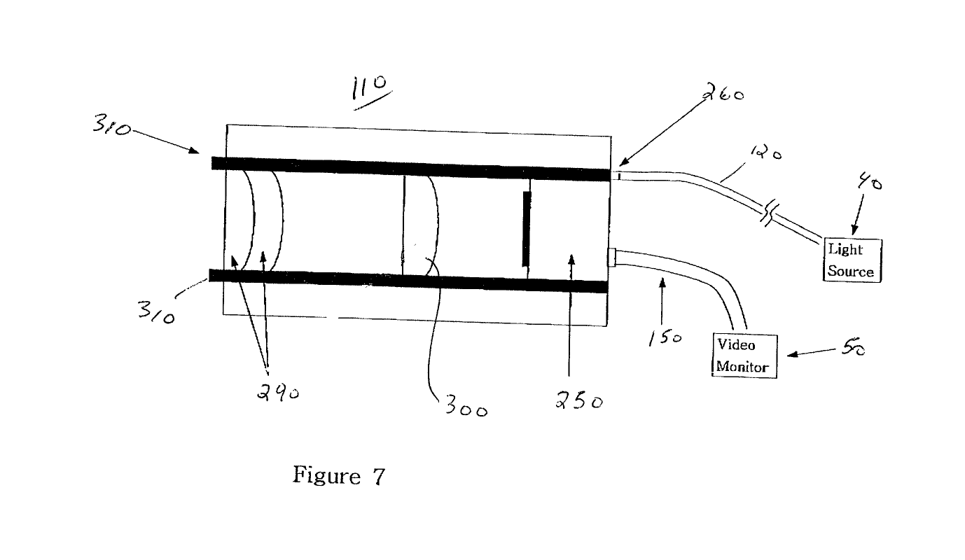

There are numerous alternate schemes to provide the illumination of the target area, and FIGS. 7-10, illustrate some of the variations within the scope of the present invention. In order to illuminate the object or surgery area, one or more fiber optic bundles 310 are shown in FIG. 7. These fiber optic bundles 310 are placed in groups around the camera lenses 290 and have an optical connector 260 that attaches to the light cable 120 that connects to the light source 40.

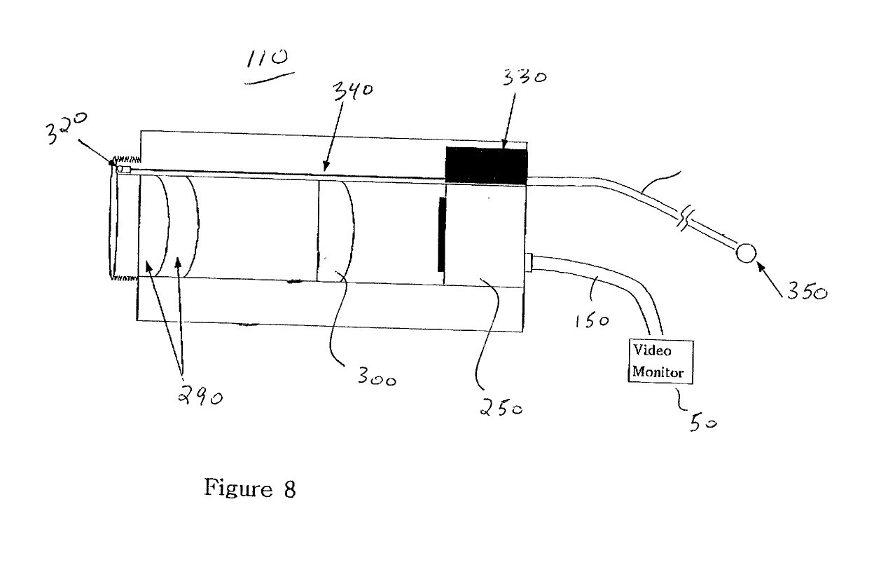

FIG. 8 illustrates a small bulb 320, such as a halogen or xenon, which is placed around the camera lenses 290. There can be a plurality of bulbs 320 placed around the lenses 290 to provide sufficient lighting. The bulb 320 connects to a battery unit 330 through electrical wire 340. The battery unit 330 in this embodiment is a 3-5VDC unit that can be connected to AC or DC converter 350 to use in-house electricity. In a working embodiment the light source is a 100 Watt halogen lamp and adjustable in intensity.

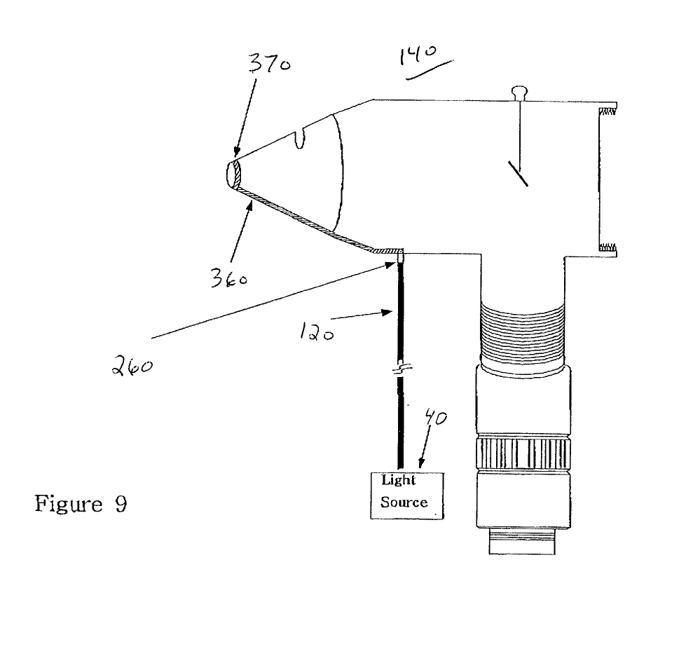

A further embodiment is shown in FIG. 9, wherein optical fibers 360 are placed in the reflection mirror housing 140. The light source 40 uses a light cable 120 to interconnect to a light connector 260 that attaches to optical fiber 360 in the front end of the housing 140. If the lighting is insufficient, the optical fibers 360 are formed into a rounded shape 370 at the end of housing.

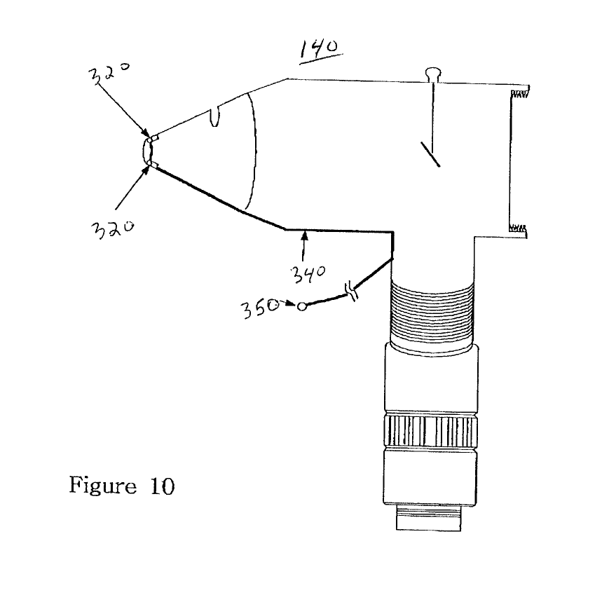

And yet another variation of an illumination means is shown in FIG. 10, wherein one or more small bulbs 320 are placed in the front end of the reflection mirror housing 140. The bulb(s) 320 are connected to a power source 350 such as a battery or AC to DC converter. The number of bulbs required is determined by the lighting requirement and the light output of the bulbs 320.

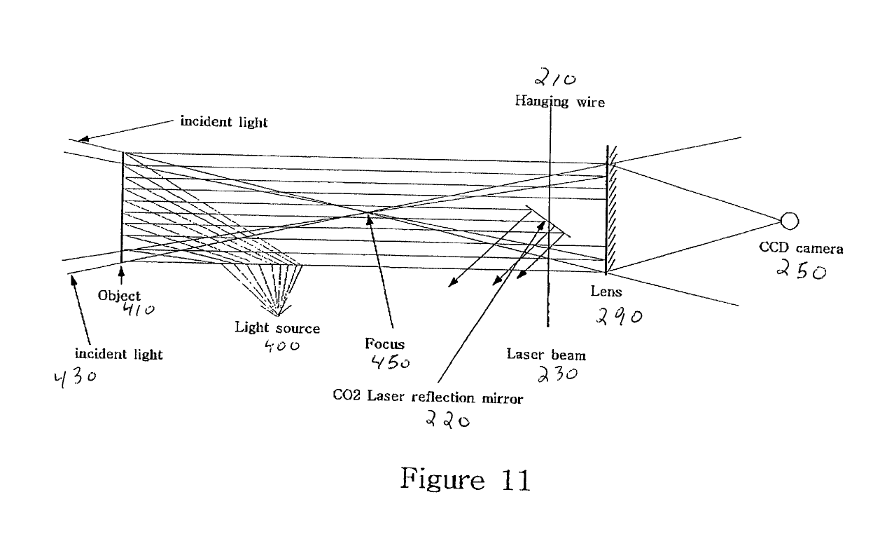

The lens theory that allows the camera to capture an image of the target area, even with the reflection mirror obstructing some portion of the path, is explained by the illustrations in FIGS. 11, 12 and 13. The camera 250 is focused for the reflections from the object, and extracts the image from the received reflections. For example, looking straight ahead and focusing on reading this document your eyes establish a focal point that is optimal for that distance. If you place your index finger directly in front of your eyes within a few inches of your nose. Despite the fact that your finger is directly in the line of vision, you can read the sentences as if the finger was not there because your eyes receive the reflected images around your finger. This principle is well-known in photography and the visual arts and adjusting the focal point allows the camera 250 to capture the surgical area during the procedure with the reflection mirror 220 directly in the line-of-sight of the camera 250.

In FIG. 11 the reflection mirror 220 and hanging wire 210 are rearward of the focal point 450. The light source 400 illuminates the object 410 and some of the incident light 430 strikes the reflection mirror and is not received by the lenses. But, some of the reflected light is received by the lens 290, and the camera 250 can discern the focused image of the object. The light source 400 in this example is used for illustrative purposes and the light source can be from any of the locations described herein.

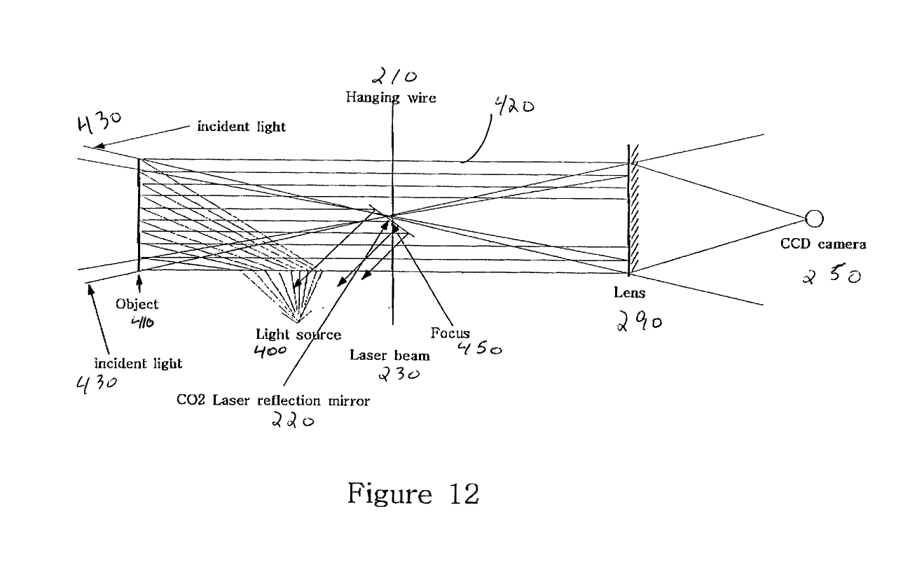

As the reflection mirror 220 is moved further from the lenses 290 and is placed at the focal point 450 as shown in FIG. 12, the lens 290 only receives the image of the reflection mirror 220 and the object is obscured. In our previous example, this would be analogous to placing your finger directly on the page and obscuring the target area.

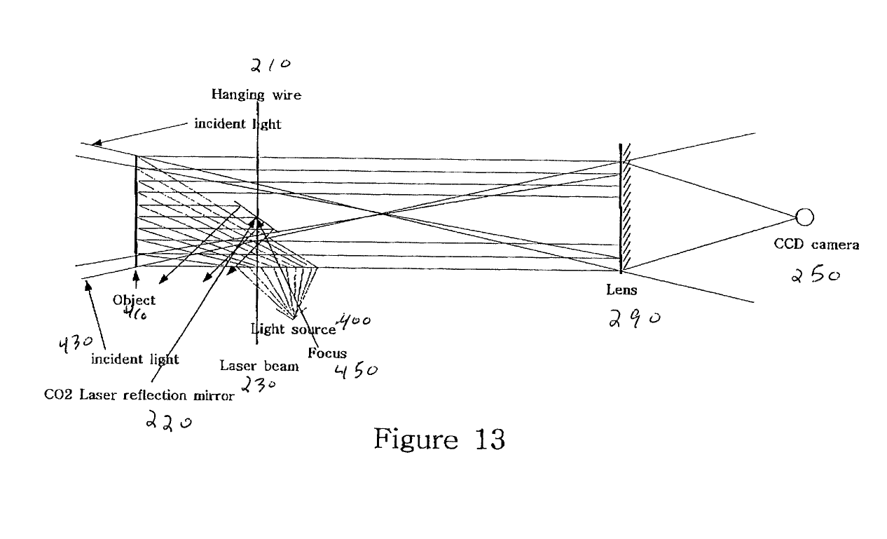

The final option is to place the obstruction, the reflection mirror 220, away from the focal point 450 and closer to the object as shown in FIG. 13. In this scenario, the object may appear as an unfocused image as the reflection mirror obstructs a larger percentage of the reflection of the image.

There are several ways to alter the focal point, including moving the lens and moving the camera. By analogy, a photographer can move towards the photo object or away from the photo object to adjust the focal distance and capture the image. Alternatively the photographer can adjust the lens of the camera to alter the focal distance.

Thus in a preferred embodiment the lens is adjusted to place the focal point according to the position illustrated in FIG. 11 by moving the camera 250. In this manner the reflection mirror 220 will not interfere with the captured image and a clear image can be observed simultaneous or live with the laser surgery.

The present invention permits the CO2 laser beam delivery, and at the same time captures the image. Referring to FIGS. 4A, 4B and 5A, the reflection mirror 220, the adjustable hanging wire 210, and the fixed wire 212 do not interfere with the image captured by the camera. The reflection mirror is a small object, located close to a camera lens 290, and the focal point is arranged to focus on the target area.

The present apparatus and methodology are distinguishable from the prior art methods that use different channels for the laser and the viewing means, or to use dichroic beam combiner to pass imaging light and laser light as the same time. Depending upon the physical location of the components of the laser head, the camera 250 is adjustable to orient the focal point to the optimal viewing position. As shown in FIG. 1, the spatial relationship of the elements allow the image to be captured with minimal interference from the reflection mirror.

In operation the laser head is moved into close proximity to the patient and the articulated arm moves to accommodate the position. The proper disposable cap is positioned onto the laser head. The laser source, video monitor, and light source are powered on and the laser head is placed in the proper position for the surgical procedure. The lens is adjusted so the image is displayed on the monitor. With the laser in a low power mode, the reflection mirror is adjusted to place the laser beam in the correct position. The zoom assembly is also adjusted to properly focus the laser beam. Finally, when all adjustments are finalized, the laser power is set to the appropriate level for the surgery. The surgeon monitors the procedure on the video display during the procedure and performs the required procedure.

The objects and advantages of the invention may be further realized and attained by means of the instrumentalities and combinations particularly pointed out in the appended claims. Accordingly, the drawing and description are to be regarded as illustrative in nature, and not as restrictive.

What is claimed is:

1. A laser beam delivery apparatus with a video display system, comprising:
a housing for said laser beam delivery apparatus, wherein said housing has a front section, a center section, and a rear section;
a laser source for generating a laser beam, wherein said laser source interconnects to said center section;
a means of directing said laser beam to said target area;
a light source for illuminating said target area;
a means for capturing an image, wherein said means for capturing is located in said rear section and wherein said means for capturing has an adjustable focal point within said center section; and
a display monitor providing said video display of said target area.

2. A laser beam delivery apparatus according to claim 1, further comprising a zoom assembly for controlling said laser beam.

3. A laser beam delivery apparatus according to claim 1, wherein said laser source is a CO2 laser.

4. A laser beam delivery apparatus according to claim 1, further comprising a disposable cap on said forward section, wherein said cap has a narrow profile.

5. A laser beam delivery apparatus according to claim 1, further comprising an articulated arm connecting said laser source to said center section.

6. A laser beam delivery apparatus according to claim 1, wherein said means of directing said laser beam is a reflection mirror.

7. A laser beam delivery apparatus according to claim 6, wherein said reflection mirror is adjustable.

8. A laser beam delivery apparatus according to claim 1, wherein said means for capturing comprises a plurality of lenses and a camera.

9. A laser beam delivery apparatus according to claim 1, wherein said light source is a bulb.

10. A laser beam delivery apparatus according to claim 1, wherein said light source is a plurality of optical fibers.

11. A laser beam delivery apparatus with a video display system, comprising:
a housing containing a laser reflection section, an image capture section, and an illumination means, wherein said illumination means provides illumination to a target area;
a laser source for generating a laser beam, wherein said laser source interconnects to said laser reflection section and said laser beam is directed to said target area;
a camera and one or more lenses within said image capturing section with an adjustable focal point; and
an output product of said target area while operating said laser.

12. A laser beam delivery apparatus according to claim 11, wherein said adjustable focal point is adjusted by moving said camera.

13. A laser beam delivery apparatus according to claim 11, wherein said output product is a video display.

14. A laser beam delivery apparatus according to claim 11, further comprising a reflection mirror for directing said laser beam, wherein said reflection mirror is suspended by wires, and wherein one of said wires is adjustably connected to a manual control.

15. A method of performing a laser surgery procedure with simultaneous video display comprising the steps of:
turning on power to a light source and a video monitor;
powering a laser source to a low-power level;
extending an articulated arm to move a laser head in close proximity to a surgery target area;
adjusting a focal point of said target area;
capturing said video display of said target area with a camera;
rotating an adjustment knob of a reflection mirror to direct a laser beam to said target area;
focusing said laser beam and adjusting power of said laser power source for said surgery procedure.

16. A method according to claim 15, wherein said step of adjusting said focal point is moving said camera, and wherein said focal point is adjusted forward of said reflection mirror.
